(19) United States
(12) Patent Application Publication
(10) Pub. No.: US 2009/0240124 A1
(43) Pub. Date: Sep. 24, 2009

(54) Systems and Methods for Measuring the Concentration of Analytes in the Human Eye

(76) Inventor: John Hefti, San Francisco, CA (US)

Correspondence Address: CLIFFORD B. PERRY, 132 N. EL CAMINO REAL, No. 347, ENCINITAS, CA, 92024-2801, US

(21) Appl. No.: 12/406,775

(22) Filed: Mar. 18, 2009

Related U.S. Application Data
(60) Provisional application No. 61/037,594, filed on Mar. 18, 2008.

(51) Int. Cl. A61B 5/1455

(52) U.S. Cl. 600/319

(57) ABSTRACT

A method for determining the concentration of an analyte within a volume located between the cornea and the iris of an eye includes determining an index of refraction of a volume disposed between a cornea and an iris of an eye, the volume including an analyte. Based upon the index of refraction of said volume, the concentration of the analyte within the volume is determined.

### CROSS REFERENCE TO RELATED APPLICATIONS

This application claims the benefit of priority of U.S. Provisional Application No. 61/037,594 filed Mar. 18, 2008, the contents of which are hereby incorporated by reference for all purposes.

### BACKGROUND

The present invention relates to systems and methods for measuring analyte concentrations, and more particular, to systems and methods for measuring analyte concentrations in the human eye.

Diabetes mellitus is a very common medical condition in which glucose is ineffectively metabolized. Historically, diabetes has been divided into two types: type 1 diabetes (also known as juvenile onset and insulin-dependent diabetes) is characterized by the loss of the ability to produce insulin, and type 2 diabetes (also known as adult unsent or non-insulin-dependent diabetes) is characterized by insulin resistance with the attendant loss of the ability to absorb glucose from extracellular fluids (such as blood) and transfer to intracellular regions. It is found clinically that many type 2 diabetics can advance to insulin-requiring status, and so the division between the two types of diabetes is not always well defined.

Diabetes is associated with many health problems, including significantly increased risks of vascular disease, neuropathy, infection and poor wound healing, blindness, renal impairment, and a higher than average rate of morbidity and premature death.

Commonly, diabetes is managed by patient self-monitoring of blood glucose levels and administering medication as needed. Many clinical studies have shown that tight glycemic control results in significantly improved health for diabetic patients. One important factor in maintaining good glycemic control is regular monitoring of blood sugar. The recognition of the importance of tight glycemic control has resulted in considerable effort to develop glucose monitoring technologies which are convenient and as painless as possible for the diabetic patient. The current standard for glucose self-monitoring involves extraction of a small amount of blood, usually from the finger tip, followed by application of the extracted sample to a test strip and subsequent analysis by a portable electronic system. Although this approach provides reasonably accurate measurements of blood glucose levels, and can be used outside of a medical setting, there are considerable disadvantages to this method. To begin with, extraction of the blood sample is painful, especially so if the sample is taken from the highly innervated areas of the finger tips. The process is also inconvenient in that it usually requires the patient to perform a number of steps in sequence, including swabbing the extraction site with an antiseptic, lancing the skin, squeezing the sample out, applying it to the test strip, blotting the test strip, inserting it into the electronic analysis unit, and awaiting the measurement. It requires patients to carry around a kit comprised of all the necessary components to self-test, and also usually requires complete cessation of other activities for a period of time while the test is administered. In addition, testing outside the home often requires that the diabetic patient administer the test in a public setting, where the patient has to acknowledge his or her disease to others. In many situations, even finding a suitable place for self-testing is a challenge. The cost of the test strips can also make regular testing a challenge. Because of the complexity, pain, cost, and psychosocial impediments to glucose self-testing, many diabetic patients find it very difficult to check blood sugar levels with sufficient frequency to maintain adequate glycemic control. Unfortunately, this has direct and unfortunate health consequences.

As a result, there have been many attempts to develop a method for detecting relevant glucose levels that do not require extraction of blood and are simple and easy to use for the patient. Many of the methods involve the use on spectroscopy, wherein specific spectral properties of glucose are used to modulate an interrogating signal and such modulation is then used to extract the glucose concentrations. There are two major challenges with most of these approaches: Glucose has relatively weak interactions with wavelengths of light that can penetrate tissues, and most tissues are very complex. In addition, the propagation of light through most tissues involved many interactions which can modulate the light in ways that do not depend on glucose. For this reason—and the fact the physiologic glucose exists is relatively small concentrations compared to other constituents—the captured signal contains many signal artifacts that mask or otherwise confound the glucose response. This is especially true for absorption modes of spectroscopy, where the signal takes the form of specific reductions in intensity due to glucose absorption. In order to measure this signal, it is necessary to discern very small changes in the intensity of the captured signal; and these changes are usually set against the backdrop of much stronger signal fluctuations due to reflection at interfaces and variability of other absorbent substances within the site of measurement.

A number of methods have employed the eye as a measurement site, since its optical properties are simpler and more predictable than skin or other measurement sites. Often these methods use either near-infrared or mid-infrared light, combined with absorption, Raman scattering, polarization, or tomographic methods. Although in the abstract these are often well-reasoned approaches, there are many difficulties with transitioning these laboratory methods to the human patient in less well-controlled situations. Despite its relative optical simplicity, the human eye is still far more complicated than standard laboratory analysis systems, and the volume of interrogation in the eye often cannot be as well controlled as an in vitro sample in the laboratory. In the case of absorption spectroscopy, reproducibly interrogating the same sample volume is a challenge, leading to measurement artifacts based variation in optical path length and sample volume. In addition, the cornea/air interface is a large source of variation in reflection and transmission, due in part to the highly variable moisture content and temperature of the superficial structures, so that getting an accurate measurement of the differential absorption is especially challenging. For polarization-based approaches, the birefringence of the cornea leads to large motion artifacts when attempting to measure glucose by its optical rotation properties. For almost all of these approaches the position of the measurement apparatus with respect to the eye is critical, and poses significant challenges to creating a practical instrument that can be used by patients outside the medical setting and going about their daily routines.

### SUMMARY

In accordance with one embodiment of the present invention, a method for determining the concentration of an analyte within a volume located between the cornea and the iris of an eye is now described. The method includes determining an index of refraction of a volume disposed between a cornea and an iris of an eye, the volume including an analyte. Based upon the index of refraction of said volume, the concentration of the analyte within the volume is determined.

The following refinements may be optionally included, either alone or in combination with several of the other refinements.

In one refinement, the act of determining an index of refraction of a volume includes the act of determining a measured index of refraction of a volume which is relative to a reference index of refraction, this act of determining a measured index of refraction including (i) detecting, at a first pixel of a light detector, a light signal from one or more features located below the cornea of the eye, wherein the volume comprises an analyte of a known concentration, (ii) detecting, at a second pixel of the light detector, a light signal from one or more features located below the cornea of the eye, wherein the volume comprises an analyte of an unknown concentration, and (iii) determining the measured index of refraction of the volume based upon the distance between the first and second pixels.

Further optionally, the aforementioned act (ii) includes detecting at the second pixel a light signal received from a first feature located below the cornea of the eye, wherein the volume includes an analyte of an unknown concentration. In this refinement, the method may further include detecting, at a third pixel of the light detector, a light signal received from a second feature located below the cornea of the eye, wherein the volume includes an analyte of said unknown concentration, and wherein determining the measured index of refraction of the volume includes (a) obtaining a weighted average between the second and third pixels, and (b) determining the measured index of refraction of the volume based upon the distance between the first pixel and said weighted average.

Also optionally, the first recited act of determining the concentration of the analyte may include: (i) obtaining a plurality of reference correlations, each reference correlation describing a correlation between an index of refraction of the volume between the cornea and the iris of the eye and a known concentration of the analyte within said volume, (ii) comparing the measured index of refraction to the index correlation of each of the plurality of reference correlations, and (iii) determining as the concentration of the analyte within said volume, the concentration of the analyte of the reference correlation having an index of refraction which is closest to the measured index of refraction.

In particular refinements of the invention, the volume is the aqueous humor of the eye, and the analyte whose concentration is to be determined therein may be glucose or other analytes, such as ascorbate, lactate, ethanol or urea. Also optionally, the one or more features may be phantoms which are projected onto the iris of the eye. Alternatively the one or more features are anatomical structures which are disposed on the iris of the eye.

Other aspects and features of the invention will be better understood in view of the following description of drawings.

### BRIEF DESCRIPTION OF THE DRAWINGS

FIG. 1 illustrates refraction for a system comprised of two homogeneous media and a flat interface between them according to the present invention.

FIG. 2 illustrates a system operable to measure the concentration of an analyte within a volume as a function of the index of refraction according to the present invention.

FIG. 3 illustrates a system of two media and a curved interface between them according to the present invention.

FIG. 4 illustrates the index of refraction of water containing glucose at various concentrations according to the present invention.

FIG. 5 illustrates the shift in the detector position of light received from a phantom over a range of glucose concentration according to the present invention.

FIG. 6 illustrates a shift in the distribution of intensities across a linear array of pixels in a light detector according to the present invention.

FIG. 7 illustrates an embodiment of the present invention in which a plurality of phantoms is used to determine spatial location shifts.

FIG. 8 illustrates an exemplary array of projected phantoms in accordance with the present invention.

FIG. 9A illustrates an exemplary system operable to measure analytes in the human eye in accordance with the present invention.

FIG. 9B illustrates a further exemplary system operable to measure analytes in the human eye in accordance with the present invention.

FIG. 9C illustrates front and side views of the orientation of the detector with respect to the eye in accordance with the present invention.

FIG. 9D illustrates an example of a pattern used to determine relative position of the detector with respect to the eye in accordance with the present invention.

FIG. 10 illustrates a cross section of a cornea and adjacent sclera in accordance with the present invention.

FIG. 11 illustrates an example of the shift in position of phantoms reflected off a cornea as a function of distance between a detector and a cornea in accordance with the present invention.

FIG. 12 illustrates a method for measuring an analyte within a volume of the human eye in accordance with the present invention.

FIG. 13 illustrates an exemplary embodiment of an operation of FIG. 12 in accordance with the present invention as described above.

For clarity, previously described features retain their reference indices in subsequent drawings.

### DETAILED DESCRIPTION OF EXEMPLARY EMBODIMENTS

Several aspects of the invention described herein is based on establishing correlations between measurable physical properties of the anterior eye and presence of certain chemical species within the eye. The method makes use of well-known methods in imaging of biologic tissues to identify features and structures in the anterior eye. These features and structures are then used to establish the location of the measurement site as well as determine relative changes in the optical properties of the measurement site. When these measurable properties of the eye are initially correlated to known values of one or more analytes of interest, these correlations can be used in subsequent measurements to establish the quantity or concentration of analytes based only on the measurement of the relevant physical property or properties, or changes therein.

The invention described herein represents a major improvement in the care of diabetes. It is able to determine glucose levels in diabetic patients without requiring any extraction of blood or other biological sample material. The measurement can be performed quickly, easily and discreetly. Part of the invention involves calibrating the measured signal for position of the instrument with respect to the eye, so that patients can use the instrument easily in the outpatient setting. Because the invention does not substantially involve the measurement of small intensity changes in the captured signal, the complexity of the apparatus is relatively simple and be made compact and highly portable. Further, the instrument can be manufactured inexpensively and without the additional costs of disposable components such as test strips, lancets, and alcohol swabs.

### DEFINITION OF TERMS

Analyte: A molecular, biochemical, or metabolic species whose concentration or relative abundance it is desired to measure. Examples of such substances include glucose, ascorbate, lactate, ethanol, urea, amino acids, salts, nitrogenous species, exogenous species such as drugs, and the like.

Index of refraction: A macroscopic quantity of a given material or medium defined as the speed of light in a vacuum divided by the phase velocity of the propagating wave in the medium. The index of refraction is usually dependent on the wavelength of the light. As used herein, ‘index of refraction’ may also be called ‘refractive index’ or ‘relative index of refraction’.

Light: As used herein, ‘light’ refers to electromagnetic waves, either in the visible range or other parts of the nearby spectrum, such as the infrared. The term light may refer to any wavelength of electromagnetic waves, and is not limited to wavelengths that are visible to the human eye. Examples include light in the visible range, infrared, both near, mid, and far, and ultraviolet light.

Optical axis: Defined as an axis (line segment) perpendicular to the anterior-most part of the cornea that also traverses through the pupillary center and fovea of the retina.

Phantom: An anatomic structure or externally projected feature within or nearby the measurement region used in the measurement process. Examples include one or more parts of the iris, including a majority of the iris, cornea, or other anatomic structures of the human eye.

One embodiment of invention is based on measuring optical properties of the anterior part of the eye, specifically from the surface of the cornea to the iris. This region is well-known to be in fairly close equilibrium with plasma glucose levels, and is also optically less complicated than other potential measurement sites like the skin. The bulk of this volume is comprised of the aqueous humor, a non-cellular liquid comprised of water (˜99%), ions (HCO3−, buffers, metabolic acids, Cl—Na+; K+; Ca2+; PO42−) glucose, lactate, ascorbate, amino acids, and very low densities of proteins due to ultra-filtration.

In order to appreciate the merits of the invention described herein, it is helpful to review some basic properties of spectroscopy. In general, spectroscopic analysis of a medium or material sample is based on the basic processes and properties of electromagnetic energy interacting with matter. Although the precise details of the physics involved in these processes can be quite complex, one common procedure is to subsume this complexity into a single mathematical equation which represents a wavelength-dependent macroscopic average of the interaction between the interrogating light and the medium being studied by that light. This equation is commonly known as the complex form of the index of refraction, as is well-known to those skilled in the art of spectroscopy, and is written:

η(λ)=n(λ)+iκ(λ) (1.1)

Here, λ is the wavelength of the light, n is the relative index of refraction defined as the speed of light in a vacuum divided by the phase velocity of the propagating wave in the medium being studied, λ is called the extinction coefficient and gives the (optionally normalized) absorption or dissipation of the electromagnetic energy in the medium being studied, and i is the square root of −1. To those skilled in the art of mathematics, equation 1.1 will be appreciated as representing a complex number, wherein the first term on the right hand side of the equation is commonly referred to as the ‘real’ part of the refractive index, and the second term is commonly referred to as the ‘imaginary’ part of the refractive index.

Equation 1.1, and generalizations which includes such effects as polarization, can be incorporated into a set of equations well-known to those skilled in the optical arts to describe the macroscopic properties of the interaction of light (or other non-visible electromagnetic waves) of wavelength λ (and optionally of specified intensity, phase and polarization) with a given sample as described by equation 1.1, once properties volume, geometry, reflection and transmission at interfaces of optically different media, optical path lengths, and other properties of the sample and its environs are included in the analysis.

As mentioned previously, measurement of the absorption component [κ(λ)] can be very difficult, especially for analytes that cause weak absorption due to low concentrations relative to other constituents or inherently small absorption cross sections. However, the measurement of the real part of the index of refraction does not require the ability to precisely measure intensity, and thus is largely immune from many of the above-mentioned problems. This is especially true if the desired quantity to be measured is the change in the real part of equation 1.1, not the absolute value of the real part. Moreover, given a sufficient number of wavelengths used in the measurement of the real part of the index of refraction, it is possible to determine the absorption as well by using the well-known Kramers-Kronig relationship.

This difference can be readily appreciated by considering the mathematical equations that govern refraction and absorption, corresponding to the real and imaginary components, respectively, of the complex index of refraction. Absorption is often described using the Beer-Lambert Law:

A(λ)=α[X]l (1.2)

In words, equation 1.2 states that the total absorption A as a function of wavelength λ due to a given concentration of species [X] in solution is equal to the absorption coefficient α, multiplied by the concentration of X and the path length l.

Absorption is measured in a laboratory system by providing a light source operable to provide light to a sample, usually contained in a vessel. A detector is provided to capture the light that has passed through both the vessel and the sample. The absorption due to a species X in the sample will manifest itself as a change in the intensity of the captured light. If all other optically-dependent properties of the system remain fixed, the change in the intensity of the captured light can be correlated to the change in concentration of analyte X in the sample. It is the difficulty of keeping these properties fixed—especially in complex biological structures outside the laboratory setting—that makes absorption measurements so challenging.

In the case of refraction, a commonly used equation is Snell's Law:

$$\frac{sin {θ}_{1}}{sin {θ}_{2}} = \frac{{n}_{2}}{{n}_{1}} \qquad ( 1.3 a )$$

Equat θ₁ ion 1.3a relates the direction of light propagation at the interface between two optical media and the respective indices of refraction n1 and n2 for each of the two media. θ₁ is commonly called the angle of incidence, and θ₂ is commonly called the angle of transmission, wherein said angles are defined by an imaginary line perpendicular to the tangent of the interface at the point of incidence, is well known to those skilled in the art of optics. Equation 1.3a thus states that for a given angle of incidence, the angle of transmission will depend on the relative indices of refraction according to the following equation:

$${θ}_{2} = {sin}^{- 1} [ \frac{{n}_{1}}{{n}_{2}} sin {θ}_{1} ] \qquad ( 1.3 b )$$

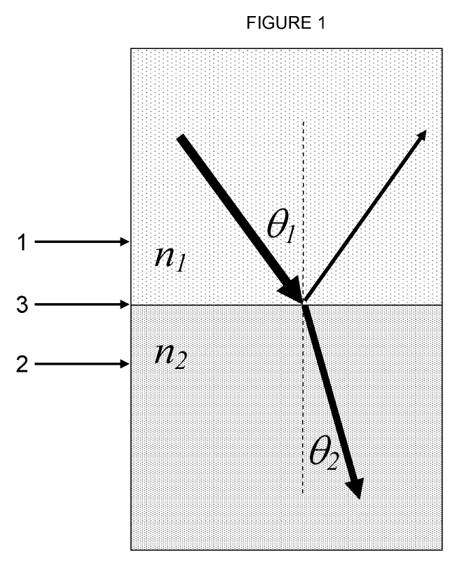

FIG. 1 illustrates refraction for a simple system comprised of two homogeneous media and a flat interface between them. Light traveling through the first medium 1 is incident on the interface 3 between the two media at an angle of incidence θ₁. The light is then refracted to a second angle θ₂, the angle of transmission, in the second medium 2. The relationship between the angle of incidence and angle of transmission is determined by the ratio of the indices of refraction n₁ and n₂ for each of the two media, multiplied by the sine of the angle of incidence, according to equation 1.3b.

If either medium contains an analyte whose concentration it is desired to measure, and further if the index of refraction for that medium varies as a function of analyte concentration, then it is possible to determine the analyte concentration by measuring the change in refraction of the interrogating light. This measurement may consist of determining the shift in the spatial location of a beam of light traversing the two media, whose direction is altered at the interface, through the use of a spatially addressable light detector (for example a charge coupled device, or CCD). One may optionally determine analyte concentration by generating a calibrated look-up table comprised of refractive values for known concentrations of the analyte and extrapolating and/or interpolating. Alternatively, one can use theoretical considerations to determine the indices of refraction.

A significant advantage of the refractive method of measurement is that, to a large degree, the precise intensity of the interrogation and captured light is not important. As long as the captured light is sufficiently intense to be distinguished from the background illumination, and its position can be measured with suitable precision, the intensity is not important. To the extent that this is the case, all of the properties of the system which affect intensity of the captured light are irrelevant to the precision and/or accuracy of the measurement.

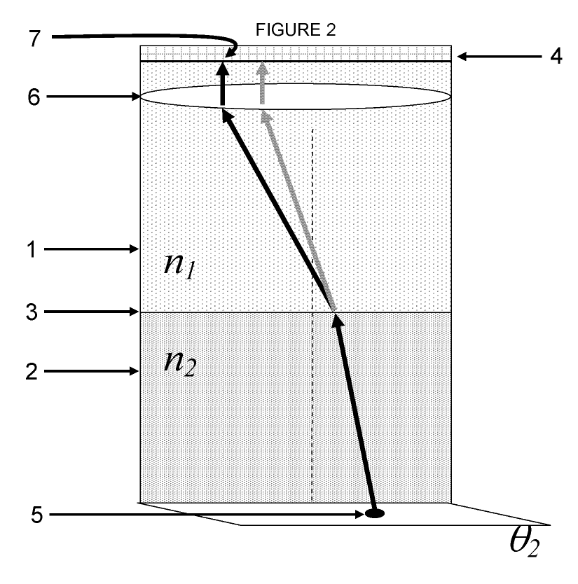

For example, one may determine the concentration of a given analyte if the refractive index n as a function of analyte concentration is known or can be determined. FIG. 2 illustrates an exemplary system operable to do this. The system includes a light source (not shown), a spatially addressable light detector 4 (for example a CCD or CMOS device), an optional focusing element 6 placed between the detector and the interface, and two optical media 1 and 2 (for example, air and an aqueous sample with index of refraction greater than that of air which contains the analyte to be measured) in contact through an interface 3. Also provided is a phantom 5, optionally either an external phantom (for example, a pattern of light projected onto a surface within medium 2) or an internal phantom (for example, an existing pattern on a surface within medium 2) whose apparent position 7 is used to determine the refractive change. The lighter shaded arrow shows the previous angle of transmission when the analyte concentration in medium 2 is of a first concentration, and the darker shaded arrow shows a changed angle of transmission due to a changing index of refraction when the analyte concentration in medium 2 is of a second concentration. That is to say, the angle that the emerging ray undergoes at the interface is a function of the difference of the indices of refraction.

The apparent position 7 of the phantom will depend on the degree of refraction at the interface 3 in FIG. 2. For example, if increasing the analyte concentration in medium 2 causes the index of refraction in medium 2 to become closer to the index of refraction in medium 1, then the angle of transmission will become closer to the angle of incidence. This will cause an apparent shift in the position of the phantom 5. By correlating this shift in position to known concentrations of the analyte, it is possible to directly measure the concentration of the analyte. Alternatively, in some cases it may be possible to determine the concentration of the analyte by using theoretical considerations that relate the index of refraction to analyte concentration.

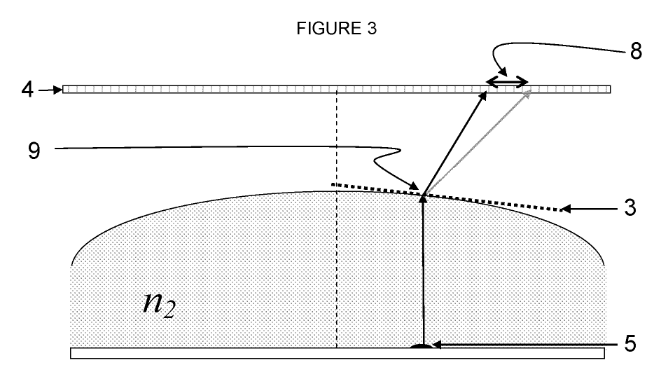

Those skilled in the art of optics will appreciate that the invention described above and in FIG. 2 can be extended and modified in many ways. For example, a system comprised of two media can include air for the first medium, an aqueous fluid for the second medium (for example, the aqueous humor of the eye), and a curved interface between them (for example, the cornea of the eye). FIG. 3 shows such a system. In this diagram, the angles of incidence and transmission are now defined by the tangent 3 of the surface at the point 9 where the ray passes through it The phantom 5 appears at the detector 4 at a spatial address that depends on the index of refraction of the aqueous medium. When the index of refraction is changed due to a change in the analyte concentration, the position of the image shifts 8. The extent of the shift in position 8 will depend on the analyte concentration.

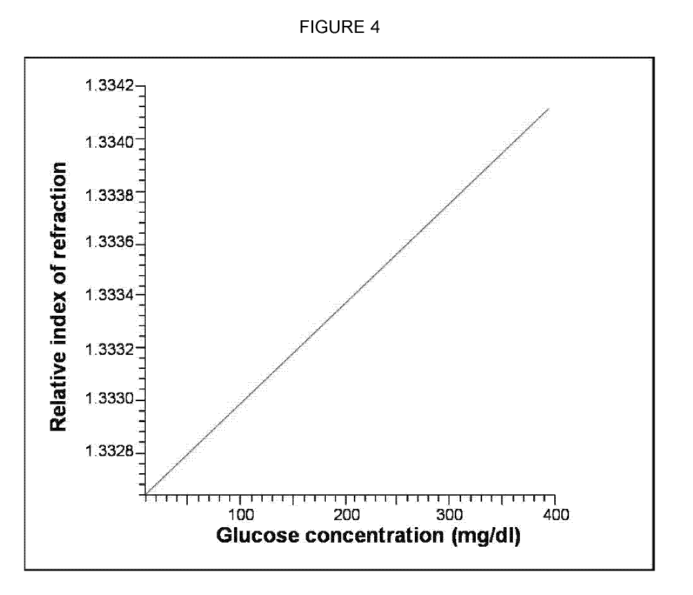

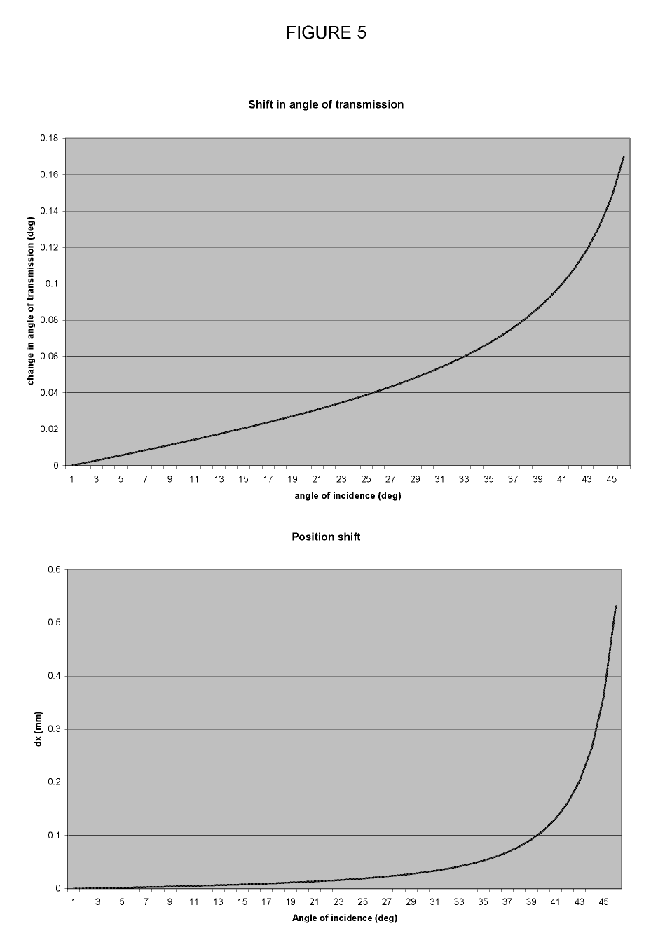

For example, FIG. 4 shows the index of refraction of water containing glucose at various concentrations. From these data, it is possible to demonstrate the shift in apparent position of a phantom as a function of glucose concentration. FIG. 5 demonstrates this shift for a detector positioned 20 millimeters from the interface, over a glucose range of 0-400 milligrams per deciliter (mg/dl). Although the actual change in the index of refraction is small, there is a distinctly measurable shift in the apparent position of the phantom, especially at the larger angles of incidence. For example, the interface may be comprised of the cornea of the eye, and the phantom may be located on the iris of the eye (optionally either an intrinsic feature of the iris or an image projected onto the iris), so that the shift in apparent position of the phantom at the detector occurs because the refractive index of the aqueous humor changes as a function of glucose concentration. If this shift is correlated to actual glucose concentration, either empirically or theoretically, the shift in position can be used to determine the concentration of glucose or other analytes.

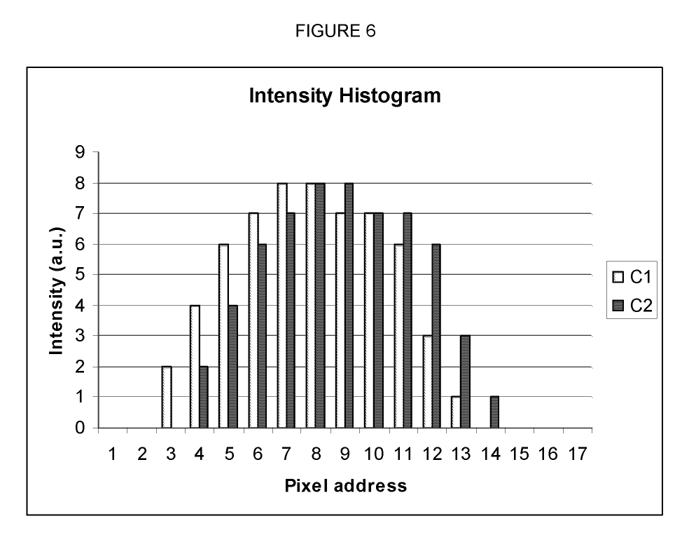

As stated previously, there are several advantages to this approach. First, so long as the phantoms are discernable from the background image, the determination of analyte concentration is largely independent of the intensity of the image. This is an important advantage, since intensity measurements are subject to a variety of artifacts, including variation in reflection and transmission at the cornea due to temperature and hydration levels, variations in source intensity and detector linearity and sensitivity, and the like. Further, since analyte concentration is mapped into spatial locations on the detector, it is possible to achieve very good resolution. For example, according to the previous example, at an angle of incidence of 40 degrees there is a 100 micrometer (μM, or 10⁻⁶ meters) shift in spatial position due to a 400 mg/dl change in glucose concentration. Using fairly simple optics it is possible to achieve 5 μM spatial resolution at the detector. This translates into a resolution of approximately 20 mg/dl of analyte concentration well within the range of accuracy required for many clinical uses. However, even greater spatial discrimination can be achieved using any of the well-known methods of statistical analysis on the intensity histograms arising from the detector. For example, FIG. 6 shows one possible shift in the distribution of intensities across a linear (one dimensional) array of pixels, resulting from the projection of the phantom onto multiple pixels of the detector. In this example, there is a small shift in the spatial projection of the phantom due to a shift in analyte concentration from C₁ to C₂. The center of the projection can be calculated using the following equation:

$$Center = \frac{{\sum }_{i} {I}_{i} {x}_{i}}{{\sum }_{i} {I}_{i}} \qquad ( 1.4 )$$

In equation 1.4, Iᵢ is the intensity at the ith pixel and xᵢ is the spatial address of the i^{th} pixel. From this it is easily calculated that the center of the projected image for concentration C₁ is approximately 7.53 and for concentration C₂ it is approximately 8.53. Using this and/or other approaches well known to those skilled in the art of imaging and image processing it is possible to resolve very small shifts in the apparent position of a phantom, and thus obtain very high measurement resolution.

It is readily appreciated that the approach described above can be extended to include a plurality of phantoms, both internal (for example one or more features of the iris, cornea, and/or other anatomic structures of the eye) and/or external (for example specific patterns projected onto anatomic structures of the eye), and extended to two dimensional detector arrays, as well as applied to the measurement of a plurality of analytes in many other media and geometries which incorporate multiple interfaces between media of differing indices of refraction. It is also appreciated that specific wavelengths of light, and pluralities thereof, may be used to obtain spectroscopic measurements as appropriate for the measurement of specific analyte concentrations. Additionally, those skilled in the art of image processing will appreciate that various methods may be used to achieve improved accuracy and/or precision

It is also clear that in some cases it is not necessary to know the exact value of the index of refraction and/or the anatomic complexity of the region under interrogation in order to make practical and useful measurements; it may suffice to know only that index of refraction will change reproducibly with respect to the change in concentration or quantity of one or more analytes in a sample, and that the anatomy of the sample is unchanged from one measurement to another. For example, if it can be determined that the index of refraction and/or absorption of a given sample changes in a reproducible way as a function of concentration of a given analyte, then this relationship can be used to determine the concentration in a given sample once the relationship is established. Moreover, if one or more measurable physical properties can be linked reproducibly to changes in the index of refraction—even if the index of refraction itself is never actually measured—then such a physical property or properties may be correlated to the presence and concentration of one or more analytes in the sample. This correlation, once established, may be used to make predictions of the concentration of said analytes at future times.

### Plurality of Phantoms

In the previous description of the invention, it was demonstrated that shifts in the spatial location of the projection of a phantom onto an addressable light detector can be used to measure the concentration of an analyte. Implicit in the description is the assumption that the detector is held in a fixed position with respect to the volume of measurement. In some cases this assumption may not be practical. For these situations, an alternative embodiment of the invention may be used in which a plurality of phantoms are used to determine spatial location shifts, wherein the shift is ascertained by measuring the relative positions of two or more phantoms with respect to one another.

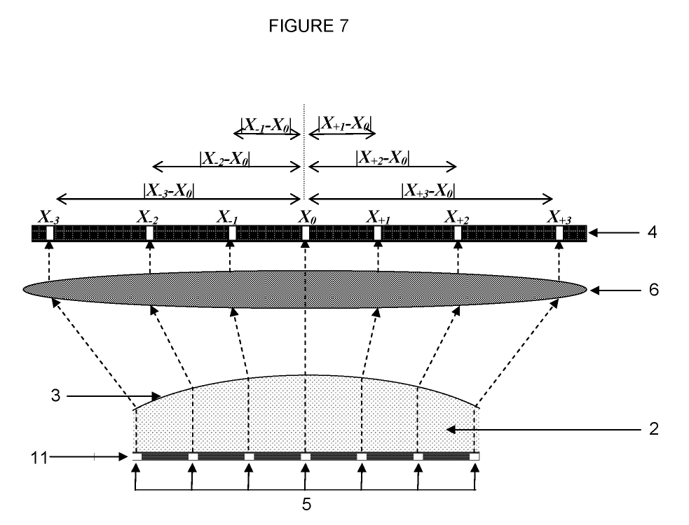

FIG. 7 shows one example of this embodiment. A plurality of phantoms, either external (such as a projected pattern of light) or internal (such as internal features of the structure 11, which may be the iris and/or cornea of the eye), is provided. Also provided is a volume comprised of a refractive medium 2 (for example, the aqueous humor of the eye) and containing one or more analytes (for example, glucose) whose concentration it is desired to measure. An imaging device 4 (for example, a CCD) and a focusing element 6 are also provided and placed at a predetermined distance from the non-planar interface 3 (for example, the cornea of the human eye).

FIG. 7 shows how the images of the phantoms propagate through the system and are captured by the detector, using ray tracing diagrams. The angle of transmission is represented by the bend in each ray at the interface 3, and is a function of the refractive index of the medium 2. Each ray then propagates from the interface 3 though the focusing element 6, where it is optionally refracted so that the image of each phantom is mapped onto a unique set of detector addresses for each phantom.

Once each phantom has its image projected onto the detector 4, the spatial location of each image is recorded. For a non-planar interface 3, the shift in spatial position will be a function of the angle between the incident ray and the tangent of the interface at the point where the ray emerges from the interface. For interfaces whose curvature changes as a function of distance from the geometric center (shown in FIG. 7 as X₀) of the interface (for example, the cornea of the eye), the spatial shift in the apparent position of a given phantom will also change as a function of the distance from the geometrical center (which may optionally be chosen to be the point on the interface 3 where the tangent is zero). This can be seen as a direct consequence of equation 1.3b, where the angle of incidence θ₁ is defined by the tangent of the interface at the point of incidence: the angle of transmission increases with the angle of incidence, and therefore the spatial shift also increases.

The distance between the spatial locations of two or more images are calculated, and then compared to known or predetermined shifts that are correlated to analyte concentrations, and a determination is made, optionally through interpolation and/or extrapolation, of the concentration of the analyte(s) of interest. Additional precision and/or accuracy can be achieved by taking the difference in spatial locations between a multiplicity of phantom images, and by using methods described previously and/or other methods well known to those skilled in the art of imaging for determining the precise spatial location of each image.

It is apparent that the approach described in this section possesses translational invariance (for example, a shift in position that is parallel to the plane of the detector interface) with respect to the detector 4, so long as the detector is positioned such that it is capable of capturing a sufficient number of phantom images to achieve the desired accuracy of the measurement. This can be seen by adding an arbitrary global translation A to each spatial location of the phantom images, then taking the difference between any two image positions Xᵢ and Xⱼ:

$${X}_{i} \to {X}_{i} + A \\ {X}_{j} \to {X}_{j} + A \\ Δ {X}_{j , i} = \vert ( {X}_{j} + A ) - ( {X}_{i} + A ) \vert \\ = \vert {X}_{j} - {X}_{i} \vert \qquad ( 1.5 )$$

In equation 1.5, A represents an arbitrary translational shift in the position of detector 4. The distance between any two points is independent of A.

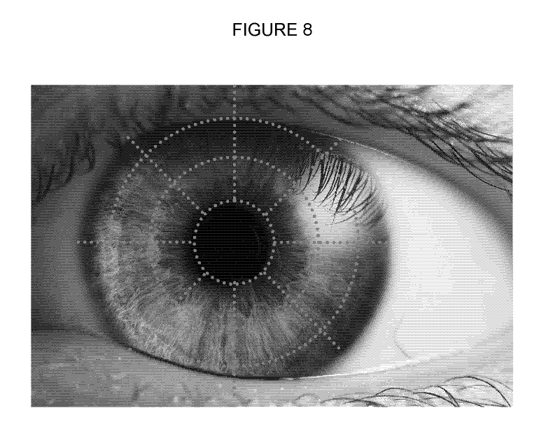

In one specific embodiment of the invention, a pattern of light is projected onto the structure 1 to create external phantoms such that the shift in the apparent position of the phantoms is maximized. For example, a set of points may be projected onto the human eye such that substantially all of the points are positioned in the region between the outer boundary of the pupil of the eye and the outer boundary of the iris (where outer is defined as regions positioned radially outward from the optical center of the cornea, which is positioned near the pupillary center). In this case, the spatial shift is maximized due to the increased curvature of the intervening portion of the cornea, according to equation 1.3b. One possible pattern of projected phantoms is shown in FIG. 8. In this case, a plurality of phantoms is projected onto the iris through the more curved parts of the cornea, so that the difference in the apparent position of the phantoms as they are imaged on the iris is maximized. FIG. 8 shows one possible pattern of projected phantoms, but there may be many different patterns of projected phantoms that could be used. In FIG. 8, a linear array of projected phantoms is placed on the iris. When the index of refraction in the aqueous humor changes due to a change in analyte concentration, the spatial distribution of each radial group of phantoms will change. The change in this distribution as a function of analyte concentration, when compared to a predetermined and/or calibrated reference, can be used to determine the concentration of the analyte. By measuring and comparing the spatial distribution of each radial group of phantoms, additional statistical power is obtained, and the accuracy of the measurement is improved.

### Multiple Interfaces: Cornea and Iris

In the previous embodiments it was shown that the concentration of specific analyte(s) can be measured in a refracting medium by using the appropriate phantom(s) and apparent spatial shifts thereof. In one embodiment of the invention, a plurality of phantoms is used to achieve translational invariance of the measurement through the use of relative differences in the spatial distribution of the phantoms. In another embodiment of the invention, it is shown how one can effect measurement of specific analyte(s) without the need for precise positioning of the detector with respect to the volume being measured. In some cases, for example measurement of ocular glucose levels by diabetic patients, it is desirable to have some flexibility in how precisely the measurement apparatus is positioned with respect to distance from the eye.

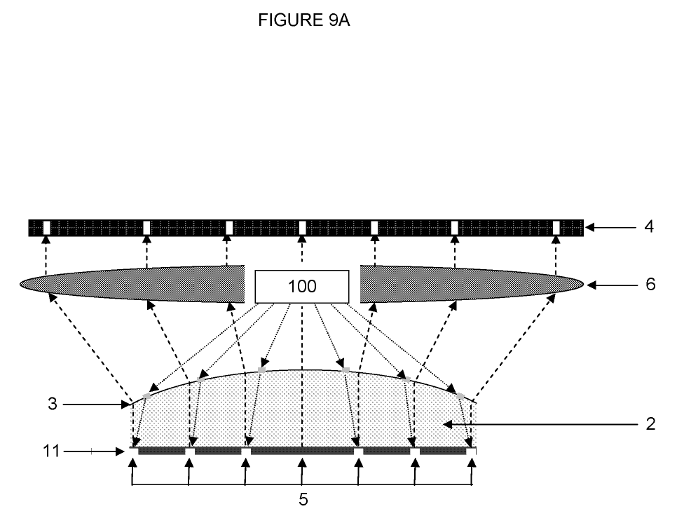

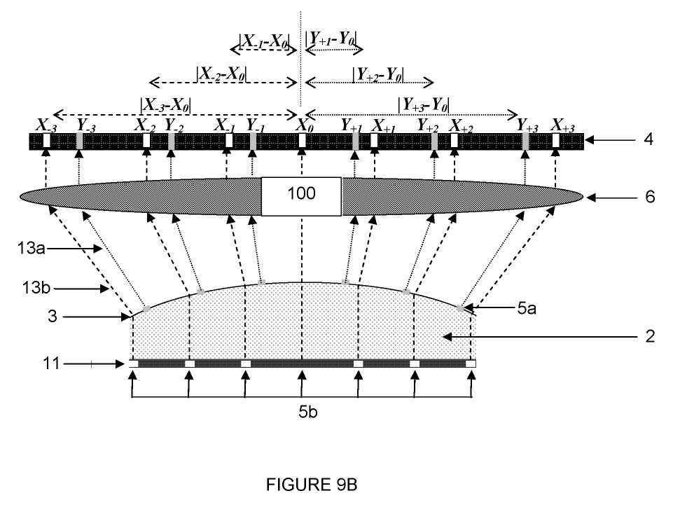

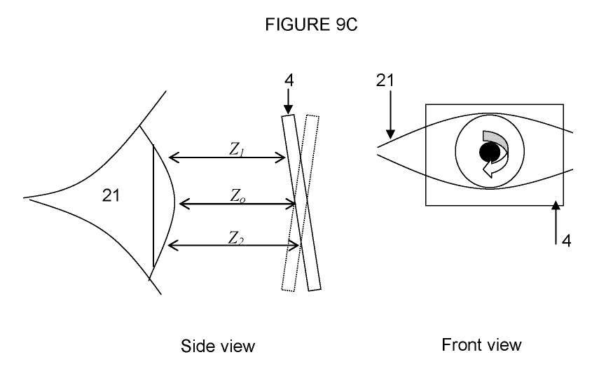

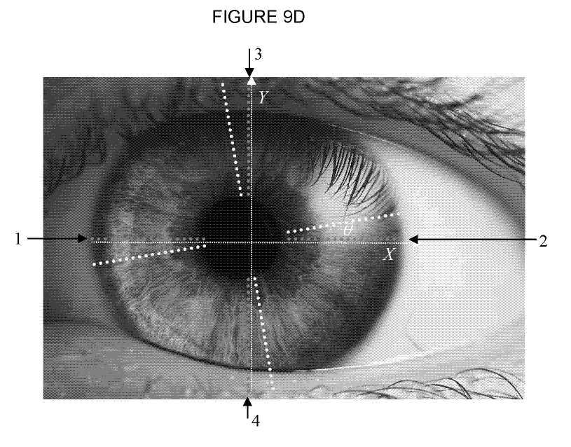

FIG. 9A shows a diagram of the general approach. A light source 100 is used to project a pattern of phantoms onto the cornea 3, which is partially reflected back to the detector and partially transmitted through the cornea and aqueous humor 2 onto the iris 11. The phantoms reflected off the cornea will depend on the corneal topography but not the composition of the underlying structures (such as the aqueous humor and/or iris). The phantoms projected onto the iris 1 will have a spatial dependence that depends on the index of refraction of the cornea and aqueous humor, as well as the original pattern and the distance from the light source to the iris. The phantoms projected onto the iris are then imaged as described previously and as shown in FIG. 7. The phantoms reflected off the interface 3 will be distributed as a function of the original pattern configuration, the topography of the interface 3, and the distance between the interface 3 and the detector 4. If the interface 3 is of a known or determinable topography (for example using the methods of corneal topography as used in the field of refractive surgery and opthalmology) and the original projected pattern is known, then the distance from the detector 4 to the interface 3 can be determined. In this way, the phantoms reflected off the interface 3 are used to calibrate the distance from the interface 3 to the detector 4. Once this calibration is established, then the spatial shifts of the phantoms projected onto the iris 1 can be used to determine the analyte concentration(s) as described previously.

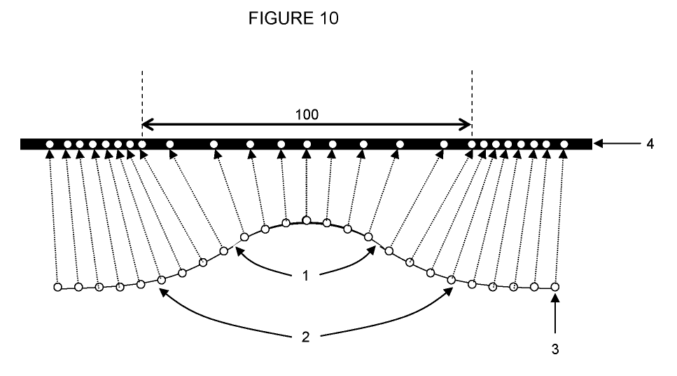

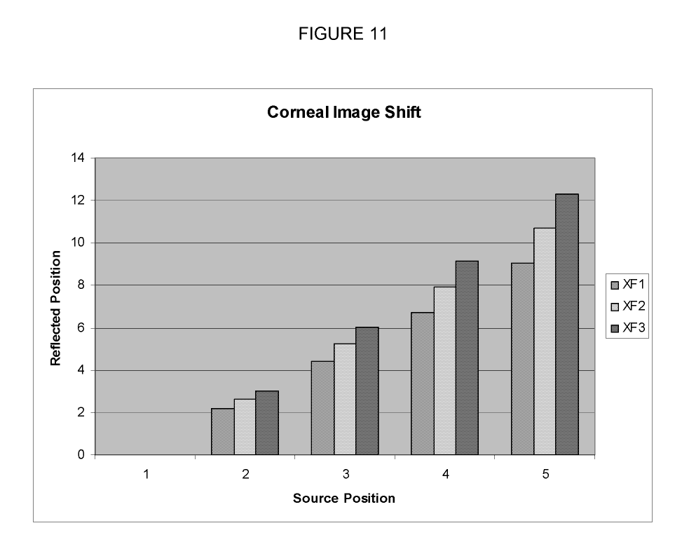

FIG. 11 shows an example of the shift in position of phantoms reflected off the cornea as a function of distance between the detector and the cornea. In this example, a projected image consisting of five phantoms spaced at one millimeter (mm) intervals in a linear configuration. Upon hitting the cornea, the angle of incidence is calculated using the tangent of the corneal surface (in this example assumed to be a parabola with quadratic coefficient of −0.02, and a linear coefficient of zero). The position of the reflected phantoms is calculated using the assumption of specular reflection and simple geometric optical considerations, for the detector placed at −15 mm (labeled XF1 in FIG. 10), −20 mm (XF2), and −25 mm (XF3) from the cornea. The captured phantoms which are reflected off the cornea show a distinct change and non-linear distribution at the detector. By inverting this relationship, the distance from the cornea can be calculated directly from the positions of the phantoms at the detector. This information can be used to calibrate the measured signal with respect to the distance the apparatus is placed from the eye.

The position of reflected phantoms off of the cornea do not depend on the index of refraction of the underlying structures, since their positions only depend on corneal topography and the position of the apparatus with respect the eye. However, it is apparent that the position of phantom images arising from the iris does depend on the index of refraction of the medium included in the volume between the cornea and iris, since the angle of transmission explicitly depends on this as stated in equation 1.3b. In addition, the apparent position of phantom images from the iris also depends on corneal topography and the distance from the iris to the detection apparatus. Thus, one embodiment of the invention, as depicted in FIG. 9B, provides a method for measuring the change in the index of refraction in the volume between the cornea 3 and the iris 11 and correlating this to glucose concentration. In this embodiment a light source 100 operable to project a specific and known pattern of phantoms onto the eye, a detector 4, and a focusing element 6 are provided, along with hardware and software capable of storing and processing the image. Known characteristics of corneal topography are combined with the measured spatial positions of a plurality of phantom images 5a arising from the cornea, whereby light signals 13a reflected from phantom images 5a are received at a detector 4 at positions shown as Y₋₃ through Y₊₃ in FIG. 9B. A first algorithm is provided such that the distribution of these images on the detector 4, when processed with the algorithm, provides a value for the distance from the cornea 3 to the detector 4. A second set of phantom images 5b are disposed on the iris 11, and light signals 13b reflected from phantom images 5b are received at the detector 4 at positions shown as X₋₃ through X₊₃ in FIG. 9B. A second algorithm is provided such that, when supplied with the distance from the cornea 3 to the detector 4 from said first algorithm and properties of corneal topography (for example, curvature and tangent thereof as a function of position), provides a value for the change in the index of refraction. This value, when correlated to a previously generated table, provides a value for the glucose concentration in the volume between the cornea 3 and iris 11.

It is also appreciated that the method just described can be applied to more than one local regions of the eye, and thus provide a means to precisely determine the position of the apparatus with respect to skew and rotation. For example, FIG. 9C shows front and side view drawings of the orientation of the detector 4 with respect to the eye 21. As shown in this figure, there are four degrees of freedom with respect to apparatus orientation: overall distance from the eye (Zₒ) and rotation of the detector 4 about each of the three spatial axes. It is shown in FIG. 9C that by measuring the distance Z₁ and Z₂ using the preciously described method, one can measure the degree of tilt in the vertical plane as shown in the side view. Although not specifically shown, using the same method in the horizontal plane one determines the tilt in this direction as well. Finally, to the extent that corneal features of a given eye are not invariant with respect to rotations about the optical axis, one determines this rotational orientation by targeting the shift in corneal topography. To the extent the cornea is rotationally invariant, so too is the precision of the measurement.

FIG. 9D shows one example of a pattern used to determine relative position of the detector with respect to the eye. Four distinct spatial phantom groups are shown, labeled 1-4. The overall distance between the apparatus (not shown) and the eye may be determined by taking an average distance for each of the four groups. The skew in the vertical plane may be determined by taking the difference of the distances between groups 1 and 2; the skew in the horizontal plane is determined by taking the difference in distance between groups 3 and 4; the rotational position is taken by determining position of the phantoms with respect to any variation in corneal topography with respect to rotations about the optical axis. Once the spatial orientation has been determined, one determines the analyte concentration using methods similar or substantially the same as described previously.

It will be obvious to those skilled in the art that there are many patterns and groupings of patterns of phantoms that may be used to obtain spatial information and analyte concentration, in keeping with the scope of the invention described herein.

For complex interfaces, such as the cornea of the human eye, the interface-reflected phantoms may be used to choose the volume to measured, as well as compensate for the distance between the measurement volume and the detector. For example, the cornea of the human eye has a characteristic curvature (usually unique for each eye) which consists of a portion of steeper curvature in the cornea and flatter curvature in the adjacent regions. FIG. 10 shows a diagram of the cross section of the cornea 1 and adjacent sclera 2. A set of phantoms 3 is projected onto the cornea 1 and the sclera 2 and is partially reflected back to the detector 6. The distribution of the phantom images on the detector 6 is a function of the initial distribution of the projected phantoms, the surface topography of the cornea 1 and the sclera 2, and the distance between the surfaces and the detector. Based on the distribution of the phantom images on the detector one chooses a region 100 in which the measurement is optimally made.

### Multiple Wavelengths

It is well known that the index of refraction of a medium is in general a function of wavelength. Further, it is also well known that chemical species and materials often interact with light in ways unique to each substance. The latter property may be used in the invention described herein to improve specificity and/or selectivity of the measurement. For example, the chemical concentrations of different species of molecules in the aqueous humor may be distinguished and measured by using wavelengths of light that interact in known and unique ways for each species to be measured. One can optionally choose wavelengths designed to interact most strongly with glucose as well as other analytes (for example ascorbate, lactate, ethanol, urea, and the like) thought to be present in the aqueous humor, and suspected of interfering with the accuracy of the glucose measurement.

One means of accomplishing this is to choose a plurality of wavelengths such that a mathematical algorithm is able to uniquely specify the concentrations of one or more analytes based on the measured response. For example, if the change in the index of refraction is known or determinable for a molecular species in the aqueous humor as a function of said species' concentration; and further, if there is a unique functional relationship between the index of refraction for a given species and wavelength, then is it possible to write a set of algebraic equations in the following form:

R_{λ}_{₁}=n₁(λ₁)[X₁]+n₂(λ₁)[X₂]+C

R_{λ}_{₂}=n₁(λ₂)[X₁]+n₂(λ₂)[X₂]+C (1.6a)

In this set of equations, R is the measured response at wavelength λ₁, n₁ is a coefficient that, when multiplied by the concentration of its respective analyte, represents the contribution to the total index of refraction due to species X₁, and [X₁] is the concentration of that species. Similar statements hold for the second analyte. C represents constant (time-invariant) background contributions to the index of refraction.

Equations 1.6a are comprised of two equations and two unknowns (namely [X₁] and [X₂]) which can be readily solved. It is appreciated that equations 1.6 can be generalized to any arbitrary number of N equations and N unknowns, as shown in equation 1.6b, and still yield a specific and unique solution for each analyte concentration. It is also appreciated that the above is exemplary and that many other mathematical and algorithmic approaches may be used, in keeping within the scope of the invention.

$${R}_{{λ}_{1}} = {n}_{1} ( {λ}_{1} ) [ {X}_{1} ] + {n}_{2} ( {λ}_{1} ) [ {X}_{2} ] + … + {n}_{N} ( {λ}_{1} ) [ {X}_{N} ] + C \\ {R}_{{λ}_{2}} = {n}_{1} ( {λ}_{2} ) [ {X}_{1} ] + {n}_{2} ( {λ}_{2} ) [ {X}_{2} ] + … + {n}_{N} ( {λ}_{2} ) [ {X}_{N} ] + C \\ ⋮ \\ {R}_{{λ}_{n}} = {n}_{1} ( {λ}_{N} ) [ {X}_{1} ] + {n}_{2} ( {λ}_{N} ) [ {X}_{2} ] + … {n}_{N} ( {λ}_{N} ) [ {X}_{N} ] + C \qquad ( 1.6 b )$$

As one exemplary embodiment of the invention, an apparatus is provided that supplies a pre-determined pattern of phantoms comprised of infrared light to the cornea, through the aqueous humor to the iris. Also provided in the apparatus is an optical focusing element (for example a lens) and a detector operable to capture the phantom images and translate them into a spatially-indexed electrical signals (for example, a CCD). Also provided is a means to transfer the image to a data storage module, for example through the use of an analog-to-digital converter placed between the detector and the storage module. A microprocessor is also provided that is able to capture and process the digital representation of the image, along with software to manipulate the image according to the previous descriptions and deliver the result of this manipulation in a user-readable format.

Using the above apparatus, the following steps are carried out:
1. Perform an initial patient-specific calibration of the apparatus according to the following steps:
   a. Determine corneal topography (for example, by using a commercial device designed for this purpose, or optionally by positioning the apparatus at varying distances from the patient's eye(s), projecting and capturing a pre-determined phantom pattern comprised of reflections off of the cornea);
   b. Store corneal topography data in an accessible look-up file;
   c. Generate a glucose calibration table by using the apparatus to calculate the refractive shift in a pre-determined phantom pattern and correlating this to one or more known values of blood glucose (for example, as determined using conventional blood draw methods); and
   d. Store glucose calibration data in an accessible look-up file.
2. Place apparatus in proximity to said patient's calibrated eye and activate image capture sequence according to the following steps:
   a. Optionally pre-focus the imager;
   b. Project phantom pattern onto eye via light source;
   c. Capture phantom image(s) via detector;
   d. Digitize image; and
   e. Store digitized image.
3. Process digitized image, according to the following steps:
   a. Identify phantom image(s) reflected from the cornea;
   b. Use algorithm to determine distance, lateral translation, rotation, and skew of the apparatus with respect to the patient's eye(s);
   c. Generate calibration table based on step 3b;
   d. Identify phantom image(s) returning from the iris;
   e. Calibrate image(s) in step 3d using calibration table generated in step 3b;
   f. Calculate change in refractive index by determining spatial shift in phantom image(s) returning from the iris;
   g. Correlate change in refractive index to glucose calibration table; and
   h. Translate correlation into glucose level (optionally via interpolation or extrapolation.

### Exemplary Alternative Embodiments

One optional method for identifying and quantifying the shift in features in the image is to use spatial frequency relationships, for example through the use of Fourier or Laplace transform methods or related methods which represent the spatial location of features in terms of a superposition of periodic functions. Other mathematical methods may include use of the Zernike polynomials to represent different components of refractive change. These methods, well-known to those skilled in the art of image analysis and ocular physiologic and function, represent the spatial location of features in an image in terms of orthogonal functions. Such methods can increase the SNR of a measurement by including or excluding certain parts of the measurement based on the relevance to the desired measurement. For example, the shift in the apparent spatial locations of features in an image may depend on the precise location of the imaging instrument. However, in a Fourier transform representation, this overall or global shift in the apparent spatial locations features can be suppressed, thus highlighting the relative shift in features with respect to one another through the shift in frequency components of the Fourier-transformed representation.

In yet other embodiments, the calibration step may optionally include preparing the eye to have the desired physiologic and functional properties. These properties may include, but are not limited to, pupil diameter, glucose content, lack of obstructing features (such as the eyelid and/or eyelashes), gaze angle, motion, accommodation, and the like. The measurement system is positioned with respect to the eye such that the desired features are visible and in suitable focus, the light source is able to effectively illuminate the eye and provide an external phantom pattern, and ambient light is sufficiently excluded from the measurement system.

Calibration may then be comprised of taking images of the eye under known (and optionally prepared) physiologic conditions within the subject, especially with respect to the analyte(s) of interest to be determined in subsequent measurements. These conditions may include blood glucose levels, levels of other physiologic constituents, such as salts proteins, nitrogen-based metabolic products, ascorbate, and the like, as determined using alternate measurements systems (for example portable or laboratory standard blood analysis systems). Images of the eye are taken under one or more known and/or prepared reference physiologic condition(s) and correlated to the physiologic condition(s).

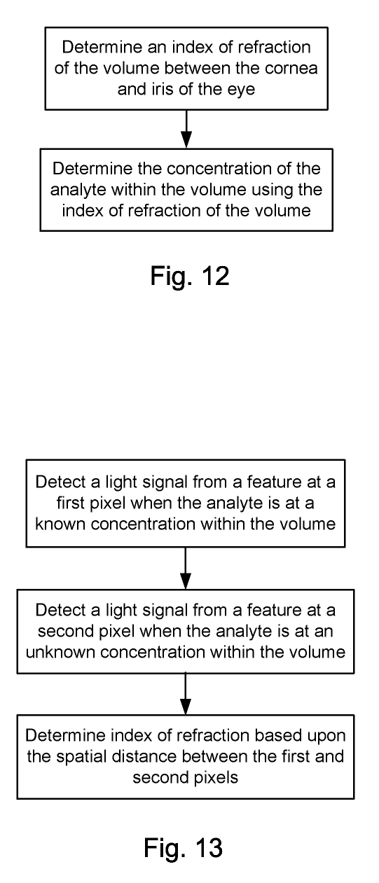

FIG. 12 illustrates a method for measuring an analyte within a volume of the human eye in accordance with the present invention as described above. At 1202, an index of refraction of a volume disposed between a cornea and an iris of an eye is determined, the volume including an analyte. At 1204, the concentration of the analyte within the volume is determined based upon the index of refraction of said volume.

FIG. 13 illustrates an exemplary embodiment of operation 1202 in accordance with the present invention as described above. In this embodiment, the operation 1202 includes operation 1302, where a light signal from one or more features located below the cornea of the eye is detected at a first pixel of a light detector, the volume including an analyte of a known concentration. At 1304, a light signal from one or more features located below the cornea of the eye is detected at a second pixel of the light detector, the volume in this instance including an analyte of an unknown concentration. At 1306, a measured index of refraction of the volume is determined based upon the distance between the first and second pixels. The measured index of refraction determined at 1306 is the index of refraction of the volume determined in operation 1202 in FIG. 12 above.

The embodiment of FIG. 13 may be expanded upon according to the embodiment described and shown in FIG. 7. For example, the second pixel of the light detector may be operable to detect a light signal received from a first feature located below the cornea of the eye, the volume including an analyte of an unknown concentration. Further exemplary, the detector may include a third detector operable to detect a light signal received from a second feature located below the cornea of the eye, the volume including an analyte of the aforementioned unknown concentration. In this embodiment, operation 1306 may include the operations of obtaining a weighted average between the second and third pixels, and determining the measured index of refraction of the volume based upon the distance between the first pixel and said weighted average.

Further exemplary, the above-mentioned methods may further include an operation of determining the distance between the detector and the cornea. Such an operation may be implemented, for example, by (i) projecting a phantom onto the cornea of the eye, the cornea have a known surface topography, (ii) detecting at a pixel within the light detector, a light signal originating from said phantom projected onto the cornea of the eye, and (iii) determining from the distance between the cornea and the detector based upon the location of the pixel within the detector and the known surface topology of the cornea.

As noted in equation (1.4) each of the pixels may be determined according to the equation:

$$Center = \frac{{\sum }_{i} {I}_{i} {x}_{i}}{{\sum }_{i} {I}_{i}}$$

where Iᵢ is the intensity at the i^{th} pixel and xᵢ is the spatial address of the i^{th} pixel.

Further exemplary of the operations FIG. 12, operation 1204 may be implemented by (i) obtaining a plurality of reference correlations, each reference correlation describing a correlation between an index of refraction of the volume between the cornea and the iris of the eye and a known concentration of the analyte within said volume, (ii) comparing the measured index of refraction to the index correlation of each of the plurality of reference correlations, and (iii) determining as the concentration of the analyte within said volume, the concentration of the analyte of the reference correlation having an index of refraction which is closest to the measured index of refraction.

Further exemplary of the invention, a system for determining the concentration of an analyte within a volume located between the cornea and the iris of an eye may as described above includes (i) a light detector 4 operable to determine an index of refraction of a volume disposed between a cornea and an iris of an eye, the volume including an analyte and (ii) means for determining the concentration of the analyte within the volume based upon the index of refraction of said volume. Further exemplary, the light detector 4 includes a first pixel operable to detect a light signal received from one or more features located below the cornea of the eye, wherein the volume comprises an analyte of a known concentration, and a second pixel operable to detect a light signal received from one or more features located below the cornea of the eye, wherein the volume comprises an analyte of an unknown concentration. Further, the means for determining the concentration of the analyte comprises a means for determining a measured index of refraction of the volume based upon the distance between the first and second pixels.

Further exemplary of the system, the means for determining the concentration of the analyte further may include (i) means for obtaining a plurality of reference correlations, each reference correlation describing a correlation between an index of refraction of the volume between the cornea and the iris of the eye and a known concentration of the analyte within said volume, (ii) means for comparing the measured index of refraction to the index correlation of each of the plurality of reference correlations, and (iii) means for determining as the concentration of the analyte within said volume, the concentration of the analyte of the reference correlation having an index of refraction which is closest to the measured index of refraction.

Further exemplary of the aforementioned system, the second pixel is operable to detect a light signal received from a first feature located below the cornea of the eye, wherein the volume comprises an analyte of an unknown concentration. The light detector 4 further includes a third pixel operable to detect a light signal received from a second feature located below the cornea of the eye, wherein the volume comprises an analyte of the aforementioned unknown concentration. In this implementation, the system means for determining the measured index of refraction of the volume includes (i) means for obtaining a weighted average between the second and third pixels, and (ii) means for determining the measured index of refraction of the volume based upon the distance between the first pixel and said weighted average.

Any one, several, or all of the system means may be implemented as a processor (central processor, embedded processor, ASIC), firm ware, or software which is operable to perform the described processes. Further exemplary, any one, several or all of the acts and operations of the methods disclosed herein may be performed using executable instruction code, that code implemented on any suitable medium (e.g. a computer readable medium such as a disk, or resident in a memory volatile, non-volatile, etc.).

The terms “a” or “an” are used to refer to one, or more than one feature described thereby. Furthermore, the term “coupled” or “connected” refers to features which are in communication with each other, either directly, or via one or more intervening structures or substances. The sequence of operations and actions referred to in method flowcharts are exemplary, and the operations and actions may be conducted in a different sequence, as well as two or more of the operations and actions conducted concurrently. Reference indicia (if any) included in the claims serves to refer to one exemplary embodiment of a claimed feature, and the claimed feature is not limited to the particular embodiment referred to by the reference indicia. The scope of the clamed feature shall be that defined by the claim wording as if the reference indicia were absent therefrom. All publications, patents, and other documents referred to herein are incorporated by reference in their entirety. To the extent of any inconsistent usage between any such incorporated document and this document, usage in this document shall control.

The foregoing exemplary embodiments of the invention have been described in sufficient detail to enable one skilled in the art to practice the invention, and it is to be understood that the embodiments may be combined. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined solely by the claims appended hereto.

What is claimed is:

1. A method for determining the concentration of an analyte within a volume located between the cornea and the iris of an eye, the method comprising:
determining an index of refraction of a volume disposed between a cornea and an iris of an eye, the volume including an analyte; and
determining the concentration of the analyte within the volume based upon the index of refraction of said volume.

2. The method of claim 1, wherein determining an index of refraction of a volume comprises determining a measured index of refraction of a volume which is relative to a reference index of refraction, comprising:
detecting, at a first pixel of a light detector, a light signal from one or more features located below the cornea of the eye, wherein the volume comprises an analyte of a known concentration;
detecting, at a second pixel of the light detector, a light signal from one or more features located below the cornea of the eye, wherein the volume comprises an analyte of an unknown concentration; and
determining the measured index of refraction of the volume based upon the distance between the first and second pixels.

3. The method of claim 1, wherein the analyte is selected from a group of analytes consisting of glucose, ascorbate, lactate, ethanol and urea.

4. The method of claim 1, wherein the volume comprises an aqueous humor of the eye.

5. The method of claim 2, wherein determining the concentration of the analyte comprises:
obtaining a plurality of reference correlations, each reference correlation describing a correlation between an index of refraction of the volume between the cornea and the iris of the eye and a known concentration of the analyte within said volume;
comparing the measured index of refraction to the index correlation of each of the plurality of reference correlations; and
determining as the concentration of the analyte within said volume, the concentration of the analyte of the reference correlation having an index of refraction which is closest to the measured index of refraction.

6. The method of claim 2, wherein the one or more features comprises one or more phantoms which are projected onto the iris of the eye.

7. The method of claim 2, wherein the one or more features comprises one or more anatomical structures which are disposed on the iris of the eye.

8. The method of claim 2, wherein said first and second pixels are determined using the equation:
$$Center = \frac{{\sum }_{i} {I}_{i} {x}_{i}}{{\sum }_{i} {I}_{i}}$$
where Iᵢ is the intensity at the ith pixel and xi is the spatial address of the i^{th} pixel.

9. The method of claim 6, wherein the one or more phantoms comprises a plurality of groups, each group comprising a plurality of linearly arranged phantoms which extend radially from a center point of the eye outwardly therefrom.

10. The method of claim 2, wherein detecting at a second pixel of the light detector comprises detecting at the second pixel a light signal received from a first feature located below the cornea of the eye, wherein the volume comprises an analyte of an unknown concentration, the method further comprising:
detecting, at a third pixel of the light detector, a light signal received from a second feature located below the cornea of the eye, wherein the volume comprises an analyte of said unknown concentration, and
wherein determining the measured index of refraction of the volume comprises:
obtaining a weighted average between the second and third pixels; and
determining the measured index of refraction of the volume based upon the distance between the first pixel and said weighted average.

11. The method of claim 2, wherein each of the first and second pixels are disposed on a light detector, and wherein the cornea comprises an interface above the volume comprising the analyte of unknown concentration, the method further comprising determining the distance between the detector and the cornea.

12. The method of claim 11, wherein determining the distance between the detector and the cornea comprises:
projecting a phantom onto the cornea of the eye, the cornea have a known surface topography
detecting at a pixel within the light detector, a light signal originating from said phantom projected onto the cornea of the eye; and
determining from the distance between the cornea and the detector based upon the location of the pixel within the detector and the known surface topology of the cornea.

13. A system for determining the concentration of an analyte within a volume located between the cornea and the iris of an eye, the system comprising:
a light detector (4) operable to determine an index of refraction of a volume disposed between a cornea and an iris of an eye, the volume including an analyte; and
means for determining the concentration of the analyte within the volume based upon the index of refraction of said volume.

14. The system of claim 13,
wherein the light detector comprises:
a first pixel operable to detect a light signal received from one or more features located below the cornea of the eye, wherein the volume comprises an analyte of a known concentration; and
a second pixel operable to detect a light signal received from one or more features located below the cornea of the eye, wherein the volume comprises an analyte of an unknown concentration; and
wherein the means for determining the concentration of the analyte comprises a means for determining a measured index of refraction of the volume based upon the distance between the first and second pixels.

15. The system of claim 14, wherein the means for determining the concentration of the analyte further comprises:
means for obtaining a plurality of reference correlations, each reference correlation describing a correlation between an index of refraction of the volume between the cornea and the iris of the eye and a known concentration of the analyte within said volume;
means for comparing the measured index of refraction to the index correlation of each of the plurality of reference correlations;
means for determining as the concentration of the analyte within said volume, the concentration of the analyte of the reference correlation having an index of refraction which is closest to the measured index of refraction.

16. The system of claim 13, wherein the one or more features comprises one or more phantoms which are projected onto the iris of the eye.

17. The system of claim 13, wherein the one or more features comprises one or more anatomical structures which are disposed on the iris of the eye.

18. The system of claim 14,
wherein the second pixel is operable to detect a light signal received from a first feature located below the cornea of the eye, wherein the volume comprises an analyte of an unknown concentration;
wherein the light detector further comprises a third pixel operable to detect a light signal received from a second feature located below the cornea of the eye, wherein the volume comprises an analyte of said unknown concentration; and
wherein the means for determining the measured index of refraction of the volume comprises:
means for obtaining a weighted average between the second and third pixels; and
means for determining the measured index of refraction of the volume based upon the distance between the first pixel and said weighted average.

19. A computer program product, resident on a computer readable medium, operable to provide executable instructions for determining the concentration of an analyte within a volume located between the cornea and the iris of an eye, the computer program product comprising:
instruction code to determine an index of refraction of a volume disposed between a cornea and an iris of an eye, the volume including an analyte; and
instruction code to determine the concentration of the analyte within the volume based upon the index of refraction of said volume.

20. The computer program product of claim 19, wherein the instruction code to determine an index of refraction of a volume comprises instruction code to determine a measured index of refraction of a volume which is relative to a reference index of refraction, comprising:
instruction code to detect, at a first pixel of a light detector, a light signal from one or more features located below the cornea of the eye, wherein the volume comprises an analyte of a known concentration;
instruction code to detect, at a second pixel of the light detector, a light signal from one or more features located below the cornea of the eye, wherein the volume comprises an analyte of an unknown concentration; and
instruction code to determine the measured index of refraction of the volume based upon the distance between the first and second pixels.
